SEQUENCE LISTING (75 sequences)

SEQ ID NO: 1 - protein, 8 aa, Homo sapiens
SSTSSWYS

SEQ ID NO: 2 - protein, 8 aa, Homo sapiens
PCIQAESE

SEQ ID NO: 3 - protein, 8 aa, Homo sapiens
DDEEIELA

SEQ ID NO: 4 - protein, 8 aa, Homo sapiens
VLEQVVTS

SEQ ID NO: 5 - protein, 8 aa, Homo sapiens
QVVQVVLD

SEQ ID NO: 6 - protein, 8 aa, Homo sapiens
KSIQEIQE

SEQ ID NO: 7 - protein, 8 aa, Homo sapiens
KDFAAEVV

SEQ ID NO: 8 - protein, 8 aa, Homo sapiens
TSYAGYIE

SEQ ID NO: 9 - protein, 8 aa, Homo sapiens
LKVAGQDG

SEQ ID NO: 10 - protein, 8 aa, Homo sapiens
FCLHGGLS

SEQ ID NO: 11 - protein, 8 aa, Homo sapiens
WMDFGRRS

SEQ ID NO: 12 - protein, 8 aa, Homo sapiens
SATAAVNP

SEQ ID NO: 13 - protein, 8 aa, Homo sapiens
RELGLGRH

SEQ ID NO: 14 - protein, 8 aa, Homo sapiens
DEGDSLDG

SEQ ID NO: 15 - protein, 8 aa, Homo sapiens
DETDMAKL

SEQ ID NO: 16 - protein, 8 aa, Homo sapiens
EECDAAEG

SEQ ID NO: 17 - protein, 8 aa, Homo sapiens
AEVDGDDD

SEQ ID NO: 18 - protein, 8 aa, Homo sapiens
DRHDGTSN

SEQ ID NO: 19 - protein, 8 aa, Homo sapiens
VEVDAPKS

SEQ ID NO: 20 - protein, 8 aa, Homo sapiens
DQTDGLGL

SEQ ID NO: 21 - protein, 8 aa, Homo sapiens
DSIDSFET

SEQ ID NO: 22 - protein, 8 aa, Homo sapiens
DDVDTKKQ

SEQ ID NO: 23 - protein, 8 aa, Homo sapiens
VEMDAAPG

SEQ ID NO: 24 - protein, 8 aa, Homo sapiens
VSWDSGGS

SEQ ID NO: 25 - protein, 8 aa, Homo sapiens
EETDGIAY

SEQ ID NO: 26 - protein, 8 aa, Homo sapiens
VETDKATV

SEQ ID NO: 27 - protein, 8 aa, Homo sapiens
GSSDPLIQ

SEQ ID NO: 28 - protein, 8 aa, Homo sapiens
DDADYKPK

SEQ ID NO: 29 - protein, 8 aa, Homo sapiens
HISSLIKL

SEQ ID NO: 30 - protein, 8 aa, Homo sapiens
DPNNLLND

SEQ ID NO: 31 - protein, 8 aa, Homo sapiens
DLSDLTAA

SEQ ID NO: 32 - protein, 8 aa, Homo sapiens
FSAYIKNS

SEQ ID NO: 33 - protein, 8 aa, Homo sapiens
EALPLLVR

SEQ ID NO: 34 - protein, 8 aa, Homo sapiens
QQGAIGSP

SEQ ID NO: 35 - protein, 8 aa, Homo sapiens
GPPGIVIG

SEQ ID NO: 36 - protein, 8 aa, Homo sapiens
MDIAIHHP

SEQ ID NO: 37 - protein, 8 aa, Homo sapiens
FFKNIVTP

SEQ ID NO: 38 - protein, 8 aa, Homo sapiens
GPLGARGI

SEQ ID NO: 39 - protein, 8 aa, Homo sapiens
HLGGAKQV

SEQ ID NO: 40 - protein, 8 aa, Homo sapiens
VWAAEAIS

SEQ ID NO: 41 - protein, 8 aa, Homo sapiens
GPLGARGI

SEQ ID NO: 42 - protein, 8 aa, Homo sapiens
ESGDYKAT

SEQ ID NO: 43 - protein, 8 aa, Homo sapiens
VAQDLNAP

SEQ ID NO: 44 - protein, 8 aa, Homo sapiens
SPDALQNP

SEQ ID NO: 45 - protein, 8 aa, Homo sapiens
PPLKLMHS

SEQ ID NO: 46 - protein, 8 aa, Homo sapiens
GPHLLVEA

SEQ ID NO: 47 - protein, 8 aa, Homo sapiens
VGPNLHGV

SEQ ID NO: 48 - protein, 8 aa, Homo sapiens
GGGNKIGP

SEQ ID NO: 49 - protein, 8 aa, Homo sapiens
LSTARFVV

SEQ ID NO: 50 - protein, 8 aa, Homo sapiens
VTEDVDIN

SEQ ID NO: 51 - protein, 8 aa, Homo sapiens
SALATTVY

SEQ ID NO: 52 - protein, 8 aa, Homo sapiens
QELISNAS

SEQ ID NO: 53 - protein, 8 aa, Homo sapiens
QELISNAS

SEQ ID NO: 54 - protein, 8 aa, Homo sapiens
WELISNAS

SEQ ID NO: 55 - protein, 8 aa, Homo sapiens
SGNYATVI

SEQ ID NO: 56 - protein, 8 aa, Homo sapiens
SIQMNVAE

SEQ ID NO: 57 - protein, 8 aa, Homo sapiens
QQNYQNSE

SEQ ID NO: 58 - protein, 8 aa, Homo sapiens
SILRLAKA

SEQ ID NO: 59 - protein, 8 aa, Homo sapiens
KFQRAITG

SEQ ID NO: 60 - protein, 8 aa, Homo sapiens
AEPKMHKT

SEQ ID NO: 61 - protein, 8 aa, Homo sapiens
TIPRAAIN

SEQ ID NO: 62 - protein, 8 aa, Homo sapiens
AEFRHDSG

SEQ ID NO: 63 - protein, 8 aa, Homo sapiens
RRKRIVGG

SEQ ID NO: 64 - protein, 8 aa, Homo sapiens
AMSRMSLS

SEQ ID NO: 65 - protein, 8 aa, Homo sapiens
PEPEQLKM

SEQ ID NO: 66 - protein, 8 aa, Homo sapiens
QSKEAIHS

SEQ ID NO: 67 - protein, 8 aa, Homo sapiens
KLKEASRS

SEQ ID NO: 68 - protein, 8 aa, Homo sapiens
RAKRSPKH

SEQ ID NO: 69 - protein, 8 aa, Homo sapiens
RKKRSTSA

SEQ ID NO: 70 - protein, 8 aa, Homo sapiens
SAMAADSN

SEQ ID NO: 71 - protein, 8 aa, Homo sapiens
TLLANINE

SEQ ID NO: 72 - protein, 8 aa, Homo sapiens
QAEETYEN

SEQ ID NO: 73 - protein, 8 aa, Homo sapiens
DVIDMSKE

SEQ ID NO: 74 - protein, 8 aa, Homo sapiens
EVLATPPA

SEQ ID NO: 75 - protein, 8 aa, Homo sapiens
APVPGTAW

(19) United States
(12) Patent Application Publication
(10) Pub. No.: US 2020/0245926 A1
(43) Pub. Date: Aug. 6, 2020

(54) COMPOSITIONS AND METHODS FOR MONITORING PROGRESSION AND REGRESSION OF DISEASE IN PATIENTS IN RESPONSE TO THERAPY

(71) Applicant: GLYMPSE BIO, INC., Cambridge, MA (US)

(72) Inventors: Sangeeta Bhatia, Lexington, MA (US); Gabriel Kwong, Atlanta, GA (US); Eric Huang, Lexington, MA (US); Sirshendu Roopom Banerjee, Lexington, MA (US); Andrew Warren, Cambridge, MA (US); Sophie Cazanave, Cambridge, MA (US)

(21) Appl. No.: 16/355,110

(22) Filed: Mar. 15, 2019

Related U.S. Application Data
(60) Provisional application No. 62/800,089, filed on Feb. 1, 2019.

(51) Int. Cl. A61B 5/00; A61K 38/48; A61K 47/10; A61K 9/00; G01N 33/50; G01N 33/574; G01N 33/68

(52) U.S. Cl. CPC A61B 5/4842; A61K 38/48; A61K 47/10; A61K 9/0019; G01N 2800/52; G01N 33/57492; G01N 33/6854; A61B 5/4848; G01N 33/5008

(57) ABSTRACT

The present invention provides method for longitudinal monitoring of disease changes and/or drug response. At multiple time points, a composition is introduced into a body of a subject (the composition includes an activity sensor comprising a plurality of reporters liberated through enzymatic cleavage, which occurs in a tissue of the body when enzymes associated with known activity of a therapeutic intervention are present in the body) and reporters are detected to determine activity levels of disease-associated enzymes in the subject, which is correlated to changes in disease state and/or drug response. A report or therapeutic response profile may further be used to support a clinical trial and for longitudinal monitoring of disease progression or regression in a subject. A prospective or active clinical trial participant, or patient undergoing treatment, may be qualified, stratified or staged based on the determined activity levels.

### CROSS-REFERENCE TO RELATED APPLICATIONS

The present patent application claims the benefit of and priority to U.S. Provisional Patent Application Ser. No. 62/800,089 filed on Feb. 1, 2019, the entirety of which is herein incorporated by reference.

### TECHNICAL FIELD

The disclosure provides methods and compositions for longitudinal monitoring of disease changes in patients in response to therapy.

### BACKGROUND

Many diseases have painful symptoms, significantly interfere with day-to-day life or are even fatal. In some cases, researchers have already discovered a drug that has the potential to cure a disease or at least alleviate the symptoms. However, before a new drug or combination of drugs is used, it should be established that the treatment is safe and efficacious.

In many important human diseases, physicians and drug developers have limited tools to assess whether a drug or combination of drugs is effective in treating disease. Drug developers often resort to histopathology, imaging, or other diagnostic methods to determine therapeutic response in patients, however these methods can take lengthy time periods (up to years) to show changes in disease progression or regression. Therefore, in clinical trials and in clinical practice, there is a need for better tools to rapidly and noninvasively monitor drug response in patients.

### SUMMARY

The invention provides methods and compositions that show disease activity in a subject and are useful for longitudinal monitoring of disease state changes and/or drug response. During longitudinal patient monitoring, methods and compositions of the invention are useful for showing efficacy of a drug or therapeutic intervention, monitoring therapeutic response over time, and detecting relapse and remission. Compositions of the disclosure include activity sensors that release detectable reporters when administered to a subject. The detectable reporters are released under certain disease-associated conditions within tissue or a body compartment of the subject. For example, activity sensors may release detectable reporters upon encountering disease-associated enzymes in the body. Detection of reporters in a sample from the subject shows the presence or stage of a disease, and can even give a measure of the rate of disease-associated activity in the subject. Tests using compositions of the invention can be administered and read non-invasively, quickly, and without requiring imaging such as x-rays.

Using compositions and methods described herein, longitudinal monitoring is performed for drug response using synthetic biomarkers and related compositions of the disclosure. Longitudinal monitoring generally involves repeated, intra-patient measurements of activity sensor velocity or changes in physiological state shown by multiple measurements over time. Repeat measurements may be used to determine if a patient's disease is stable, to show how a patient is responding to a treatment, or to reveal the effect of a treatment such as a surgical intervention. Additionally or alternatively, compositions and methods are useful in screening populations of subjects. A large population may be screened quickly and effectively to detect or rule out a particular disease state. Similarly, a cohort of disease-affected subjects can be assayed to detect and quantify an effect of a drug on the disease. Compositions and methods of the disclosure thus provide tools for supporting clinical trials and longitudinal patient monitoring of patients undergoing drug therapy. In particular, the invention allows for the rapid and inexpensive qualification of subject populations, and adaptations in clinical trial design or selection of drugs and in combinations thereof. The invention also provides methods and compositions useful for the rapid determination of subjects' response to a drug.

Compositions of the disclosure are particularly useful for longitudinal patient monitoring, precision medicine (e.g., identifying personalized treatments), patient qualification in clinical trials, defining clinical trial strategy and design of patient cohorts, as well as clinical trial go/no-go decisions involving monotherapies or combination therapies. The compositions can be administered in a minimally invasive manner and can provide informative results rapidly (e.g., in a matter of hours). Thus, the compositions provide a tool for clinicians and for screening large populations of subjects to identify a prospective cohort of trial participants, as well as clinicians to monitor disease progression or regression in subjects in a longitudinal manner.

For subjects receiving drug treatment, the composition detects activity indicative of whether the participant is responding to the treatment in a clinical trial or in clinical practice. For example, activity sensors may be designed to detect whether a tumor is growing or shrinking in size, the rate of activity or progression of the disease, whether a particular dosage of the drug is effective, and whether a participant is likely to respond to treatment.

The invention also allows for profiling of the type of response in a participant instead of merely indicating whether or not there is a response. For example, certain drugs may be directed to treatment of a disease, but the drugs may be directed to different methods of treating the disease. For example, in a disease like nonalcoholic steatohepatitis (NASH), one drug may be an anti-fibrotic drug while another is anti-inflammation or anti-NASH. Detecting the activity that the drug invokes in the body of a participant may provide insight when understanding why a participant did or did not respond to treatment.

Additionally, the invention may be used to detect whether a subject is about to respond to treatment. In an example, a subject has been diagnosed with a tumor. The subject may be treated with a checkpoint blockade drug. The subject may appear to be unresponsive to treatment because the tumor continues to grow in size after administering the checkpoint blockade. However, because of the ability to detect activity within the body, the invention may detect that the growth is from an influx of immune cells. Thus, even though the tumor has yet to decrease in size, the subject may be responsive to the treatment and a size reduction may be imminent. By sensing disease activity at the molecular level in vivo, the invention may predict responders and non-responders to drug therapy long before changes are discernable via histopathology, imaging or other prognostic methods.

According to methods of the disclosure, a composition that includes activity sensors is introduced to a subject or an active or potential participant of a clinical trial or in clinical practice by any suitable means, such as by injection. Once in the body, the activity sensor provides a plurality of reporters that are susceptible to liberation upon cleavage from the activity sensors by enzymes associated with a particular disease. The liberated reporters may then be detected, with results indicative of enzymatic activity present in the body. That enzymatic activity may be characteristic of a disease or condition, and those results may be used to qualify and stratify individuals for clinical trials and for longitudinal disease monitoring. That enzymatic activity may also be characteristic of how a participant is responding to drug treatment, which is used for monitoring of treatment and to determine efficacy in clinical trials and in clinical practice.

In certain aspects, the invention provides longitudinal monitoring methods for profiling disease activity in subjects. The methods include administering to a subject a plurality of activity sensors that include a plurality of detectable reporters that are released in the presence of disease and detecting the detectable reporters in a sample from the subject. Preferably the detection is performed at multiple time points over time. The methods are useful for longitudinal monitoring for drug response. The methods may include identifying a subject as responding to a particular treatment. That is, in personalized medicine applications, the methods may be used, e.g., by a clinician or payor to support a treatment decision. The methods may include qualifying the subject for inclusion in a clinical trial based on presence or absence of the detectable labels in the sample. The methods may further include testing members of a population using the activity sensors and identifying a first population that is affected by the disease. A report may be provided that includes summary and demographic information of the first population as a cohort for the clinical trial. The report may stratify subjects based on demographic or medical information. The report may identify subjects who will respond to therapeutic treatment. The report may further identify a second population that is not-affected by the disease, i.e., to qualify a control group for a cohort.

In some embodiments, each of the plurality of activity sensors includes a plurality of a reporter susceptible to cleavage by a specific enzyme. Accordingly, the plurality of activity sensors will release different detectable reporters depending upon what set of enzymes those reporters encounter within a subject. Preferably, the activity sensors are sensitive to a set of enzymes that include enzymes differentially expressed under a disease condition. The set may include at least one enzyme indicative of healthy tissue, useful to show a baseline enzyme activity level of the subject. The sets may include one or more enzymes indicative of one or more comorbidities to the disease. Preferably, each activity sensor comprises a carrier and a plurality of reporters. Each carrier may comprises one or a plurality of molecular subunits. Each reporter in the plurality of reporters is linked to the carrier by a cleavable linker (e.g., a peptide) containing a cleavage site of an enzyme such as a protease, wherein cleavage of one or more reporters by the one or more enzymes is indicative of enzymatic activity. In an embodiment, the carrier comprises a 30 to 50 kDa poly ethylene glycol (PEG) scaffold of covalently linked PEG subunits. In an embodiment, each reporter and cleavable linker comprises a polypeptide susceptible to cleavage by a protease known to be associated with a specific disease or condition.

Preferably, the detectable reporters are substrates for a plurality of enzymes, in which at least some of the enzymes exhibit heightened activity levels in tissue affected by the disease. In some embodiments, at least one of the enzymes does not exhibit heightened activity levels in tissue affected by the disease and is used to indicate a baseline enzyme activity level. Optionally, one of the enzymes is indicative of one or more comorbidities to a disease or condition.

In certain embodiments, each activity sensor includes a carrier comprising one or a plurality of molecular subunits and a plurality of the detectable reporters, each linked to the carrier by a cleavable linker containing a cleavage site of an enzyme. The carrier may be, for example, a 20 to 60 kDa scaffold of poly ethylene glycol (PEG) subunits (preferably about 40 kDa). Each reporter and cleavable linker may include a polypeptide susceptible to cleavage by a protease known to be associated with a specific disease or condition.

A composition of the disclosure may be introduced to an individual by any suitable method, such as injection or ingestion. After a suitable time period, which is a time period long enough for the composition to encounter enzymatic activity in the individual, a sample is collected from the individual. For example, a urine sample may be collected after about an hour. The sample is then tested by any suitable testing method, such as mass spectroscopy. Results of the testing indicate presence of specified enzymatic activity within the body, thereby providing a therapeutic profile for the individual.

Therefore, the present invention provides methods testing for enzymatic activity to determine a therapeutic profile of an individual. For example, injection of a composition designed to detect particular enzymatic activity, followed by urine sample collection after a brief waiting period, is a less time-consuming and physically demanding testing method than a liquid biopsy, tissue biopsy, or other testing method.

In certain embodiments of the methods, qualifying the subject or monitoring patient response to drug includes determining a stage of the disease in the subject. The disease may include an allergy, cardiovascular disease, degenerative disease, dietary disease, hereditary disease, immune disease, infectious disease, metabolic disease, cancer, organ disease, pregnancy, muscle injury, or trauma disease. In some embodiments, the disease is cancer. Methods may further include monitoring how a tumor in the subject responds to a drug or therapeutic treatment.

Aspects of the disclosure provide methods for determining drug efficacy. The methods may include administering—to a subject participating in a clinical trial or being treated by their physician—activity sensors comprising detectable reporters that are released from the activity sensors at a location in the body affected by a disease, detecting the reporters in a sample from the subject, and correlating a measurement of the reporters in the sample to an effect of a drug given to the subject on the disease. In some embodiments, a decrease in a quantity of certain ones of the reporters, comparted to a control from subjects administered a placebo or not currently undergoing treatment, is indicative of efficacy of the drug as to the disease. The methods optionally include determining a stage, or a change of stage, of the disease, or may include monitoring drug response over time in the subject. Methods may further include providing a report that correlates a dosage of the drug to a determined efficacy. Preferably the reports summarizes dosing information over at least a sub-population of subjects who responded to the drug.

In certain aspects, the invention provides a method for profiling disease activity in a subject. The method includes introducing—into a body of a subject—a composition comprising activity sensors that include a plurality of detectable reporters that are liberated by disease-associated enzymes. The method includes detecting the detectable reporters in a sample from the subject and including the subject in a report for a clinical trial when the detected reporters indicate a predetermined disease state in the subject. The report may include: subjects qualified for participation in the clinical trial based on presence of disease activity; subjects' responses to a drug or a placebo; subjects stratified based on demographic or medical information; other information; or combinations thereof. The report may identify subjects who will respond to therapeutic treatment. The report may be used to monitor subjects' response to a drug or placebo treatment over time.

In some embodiments, the activity sensors are introduced to the subject and reporters are detected both before the subject is administered the drug and after the subject is administered the drug. The disease may be an allergy, cardiovascular disease, degenerative disease, dietary disease, hereditary disease, immune disease, infectious disease, metabolic disease, cancer, organ disease, pregnancy, muscle injury, or traumatic injury. In certain embodiments, the disease is cancer. The method may include monitoring how a tumor in the subject is responding to a drug or therapeutic treatment.

Preferably, the detectable reporters are substrates for a plurality of enzymes, in which at least some of the enzymes exhibit heightened activity levels in tissue affected by the disease. In some embodiments, at least one of the enzymes does not exhibit heightened activity levels in tissue affected by the disease and is used to indicate a baseline enzyme activity level. Optionally, one of the enzymes is indicative of one or more comorbidities to a disease or condition.

In certain embodiments, each activity sensor includes a carrier comprising one or a plurality of molecular subunits and a plurality of the detectable reporters, each linked to the carrier by a cleavable linker containing a cleavage site of an enzyme. The carrier may be, for example, a 20 to 60 kDa scaffold of poly ethylene glycol (PEG) subunits (preferably about 40 kDa). Each reporter and cleavable linker may include a polypeptide susceptible to cleavage by a protease known to be associated with a specific disease or condition.

Methods may be used to determine a subject's therapeutic response profile by introducing a composition into a body of the subject. The therapeutic response profile may be used to monitor the subject's response to a drug. For example, the activity sensor may be introduced to the body of the subject and reporters may be detected. For monitoring purposes, the process is carried out before the subject is administered the drug and again after the subject is administered the drug.

Optionally, the therapeutic response profile indicates progression of a disease or physiological state. The physiological state or disease may comprise any suitable physiological state or disease. For example, the physiological state or disease may comprise an allergy, cardiovascular disease, degenerative disease, dietary disease, hereditary disease, immune disease, infectious disease, metabolic disease, cancer, organ disease, pregnancy, muscle injury, or trauma disease. In certain embodiment, the disease is cancer, and the present monitors how a tumor in the subject is responding to a drug or therapeutic treatment.

In an embodiment of the present invention, the composition comprises a plurality of activity sensors, each activity sensor programmed with reporters susceptible to cleavage by specific enzymes. A specific enzyme is indicative of healthy tissue, thereby indicating a baseline enzyme activity level of the subject. Another specific enzyme is indicative of a disease or condition. Other specific enzymes are indicative of one or more comorbidities to a disease or condition.

In an embodiment, the activity sensor comprises a carrier and a plurality of reporters. Each carrier comprises one or a plurality of molecular subunits. Each reporter in the plurality of reporters is linked to the carrier by a cleavable linker containing a cleavage site of an enzyme, wherein cleavage of one or more reporters by the one or more enzymes is indicative of enzymatic activity. In an embodiment, the carrier comprises a 30 to 40 kDa poly ethylene glycol (PEG) scaffold of covalently linked PEG subunits. In an embodiment, each reporter and cleavable linker comprises a polypeptide susceptible to cleavage by a protease known to be associated with a specific disease or condition.

The present invention provides methods of supporting a clinical trial and for longitudinal disease monitoring. A composition is introduced into a body of a prospective or active clinical trial participant or patient undergoing treatment for a disease. The composition comprises an activity sensor comprising a plurality of reporters liberated through enzymatic cleavage. Enzymatic cleavage occurs in a tissue of the body when enzymes associated with known activity of a therapeutic intervention are present in the body. One or more liberated reporters are detected to determine a therapeutic response profile of the prospective or active clinical trial participant or patient undergoing treatment for a disease. The prospective or active clinical trial participant—or the patient undergoing treatment—is stratified based on the therapeutic response profile.

The therapeutic response profile indicates a physiological state in the patient. In an embodiment, the physiological state comprises a disease and the therapeutic response profile further indicates staging of the disease. In certain embodiments, the physiological state comprises a pre-disease state. The pre-disease state may qualify the prospective or active clinical trial participant for a clinical trial or for ongoing monitoring of response to therapy.

In an embodiment, methods of the present invention further comprise identifying a prospective or active clinical trial participant or patient undergoing treatment who will respond to a drug or therapeutic treatment based on the therapeutic response profile. In certain embodiments, the therapeutic response profile is used to monitor a clinical trial participant's or patient's response to a drug or course of treatment by determining the therapeutic response profile before and after administering the drug or course of treatment. In an aspect, the therapeutic response profile indicates responsiveness of the clinical trial participant to the administered drug or course of treatment. In other aspects, the therapeutic response profile indicates responsiveness of the clinical trial participant or patient to a specific dose of the drug or course of treatment. In other embodiments, the therapeutic response profile indicates efficacy or effectiveness of the drug or course of treatment for the clinical trial participant or patient undergoing treatment.

### BRIEF DESCRIPTION OF THE DRAWINGS

FIG. 1 illustrates a method of longitudinal monitoring of drug response.

FIG. 2 diagrams a method according to certain embodiments.

FIG. 3 illustrates an embodiment of an activity sensor of the present invention.

FIG. 4 illustrates a method of using the present invention.

FIG. 5 shows the method for designing a composition or activity sensor cocktail.

### DETAILED DESCRIPTION

The invention provides methods and compositions that show disease activity in a subject and are useful for longitudinal monitoring of disease state changes and/or drug response. In particular embodiments, the invention is useful for longitudinal monitoring and personalized medicine as well as for recruitment, enrichment, qualification, and stratification of study participants. The invention is also useful for detecting efficacy of treatment, monitoring response to treatment over time, and detecting relapse and remission. Detection of reporters in a sample from the subject may indicate presence of a disease, stage of a disease, and a rate or a level of disease activity in the subject. Tests using compositions of the invention can be administered and read non-invasively, quickly, and without imaging by x-rays or other modalities.

Compositions of the disclosure may be used to determine subject responsiveness to a drug and efficacy of treatment. For instance, the compositions can be administered and tested at multiple time points and read to observe trends over time in disease changes or progression or remission. Regardless of whether a subject has received a treatment or the compositions are being used diagnostically, the compositions may be used to measure activity within the body. As such, the invention provides an additional understanding of the disease instead of merely the effect of the disease. Compositions may be used to study the disease and/or to study other associated activity. For example, the activity or progression of the disease may be detected, such as growth rate of a tumor. For subjects receiving drug treatment, the composition may be used to detect activity indicative of whether the participant is responding to the treatment. For example, activity sensors may be designed to detect whether a tumor is growing or shrinking in size, the rate of activity or progression of the disease, whether a particular dosage of the drug is effective, and whether a participant is likely to respond to treatment.

The invention also allows for more than a binary response of whether a participant responds to treatment. The invention provides profiling of the type of response in a participant instead of merely indicating whether or not there is a response. For example, certain drugs may be used to treat a disease. However, the drugs may be directed to different methods of treating the disease. When treating a disease like nonalcoholic steatohepatitis (NASH), one drug may be an anti-fibrotic drug and another drug may be an anti-inflammation or anti-NASH drug. Detecting activity that the drug invokes in the body of a participant provides insight as to why a participant did or did not respond to treatment.

Additionally, the invention may be used to detect whether the subject is about to respond to treatment. For example, a subject may have a tumor that has been treated or is being treated with a checkpoint blockade drug. The subject may appear to be unresponsive to treatment because the tumor continues to grow in size after administering the checkpoint blockade. However, because the invention detects activity within the body, the invention may detect that the growth is from an influx of immune cells. Thus, the subject is unresponsive to treatment, but instead is about to respond to the treatment.

The activity sensors of the present invention may detect signals generated in an organism from non-enzymatic activity. As an example, the activity may be related to conformation, and the activity sensors may detect acid, sheer, or competitive binding events. For example, the activity sensors may detect results of a mechanical event, such as leakage from red blood cells getting squeezed during cellular damage or membrane damage. As an example, a peptide may assume different conformations, or may fold differently, based on a pH change in the body.

Signals detected by the activity sensors may also indicate secondary interactions happening within the organism. The secondary interactions may be the result of events happening locally biologically. For example, signal A may indicate the activity that results in cells being killed within the body, while signal B indicates the results from the cells being killed. The activity sensors may detect signal A on its own, signal B on its own, and signals A and B together. The activity sensors provide greater sensitivity and specificity, as signals A and B may be detected together. Detection may indicate that signals A and B are happening within the same area locally in the body or that signals A and B are happening in different locations.

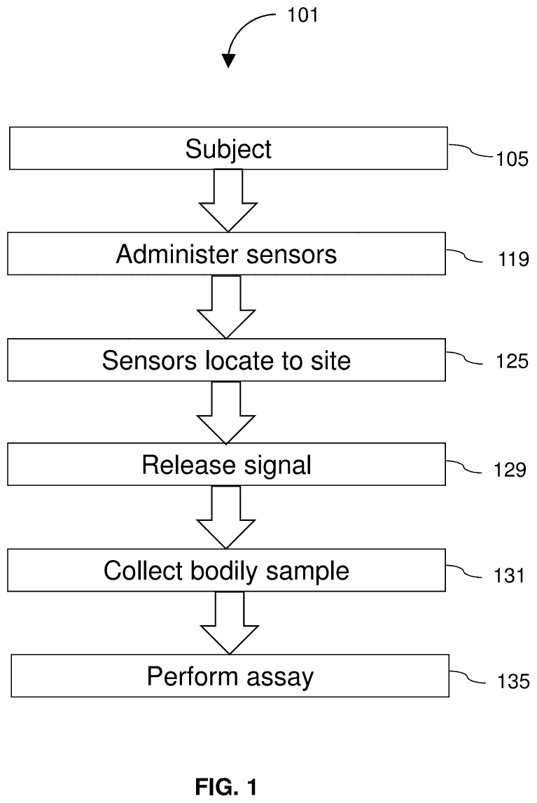

FIG. 1 shows a method 101 for longitudinal monitoring for drug response. The method 101 may be implemented in the context of a longitudinal study to determine efficacy of drugs or drug combinations. A subject is included 105 who will receive, or is receiving a treatment. Activity sensors are administered 119 to the subject. The method 101 includes monitoring change in disease condition in a subject who has received a therapeutic treatment for the disease by making measurements at multiple points over time (i.e., in a longitudinal manner). Those measurements include administering 119, to the subject, activity sensors comprising detectable reporters that are released from the activity sensors in proportion to a state of the disease. The activity sensors traffic 125 within the subject to a location in the body affected by the condition. At the location, the activity sensors release 129 a signal that can be non-invasively detected. A sample is obtained 131 from the subject, and an assay is performed 135 to detect the signal in the sample. By detecting the reporters in a sample from the subject, one may correlated quantities of the reporters to disease state. In embodiments, because multiple measurements are made over time, the method 101 provides for longitudinal monitoring of any disease state changes over time in the subject.

The method 101 preferably uses a plurality of activity sensors 21. The activity sensors 21 are used to measure disease-associated activity within the body such that levels of such activity measured using the sensors shows disease progression or that treatment is being effective. The activity sensors may be given over time to monitor a subject's response to treatment and to show whether or not the treatment is effectively treating the disease in the subject. Different drugs or combinations of drugs can be studied in different subjects or at different times to identify drugs or combinations that work well for treating a condition. The activity sensors provide a marker of health/disease progression and provide the marker very quickly (detecting a signal in a sample from the subject may be within hours of administering the activity sensors). Activity sensors can also specifically report the activity of multiple enzymes including ones for which expression is dysregulated in the disease state and others that are specific to co-morbidities. Preferably, the signal is provided by fragments of the activity sensors that are released by enzymes (e.g., extracellular proteases) for which expression is upregulated at the location in the body when affected by the condition. Using the methods for longitudinal monitoring, it may be found that a decrease in certain fragments from the subject (compared to a control receiving a placebo) is indicative of efficacy of the treatment as to the condition.

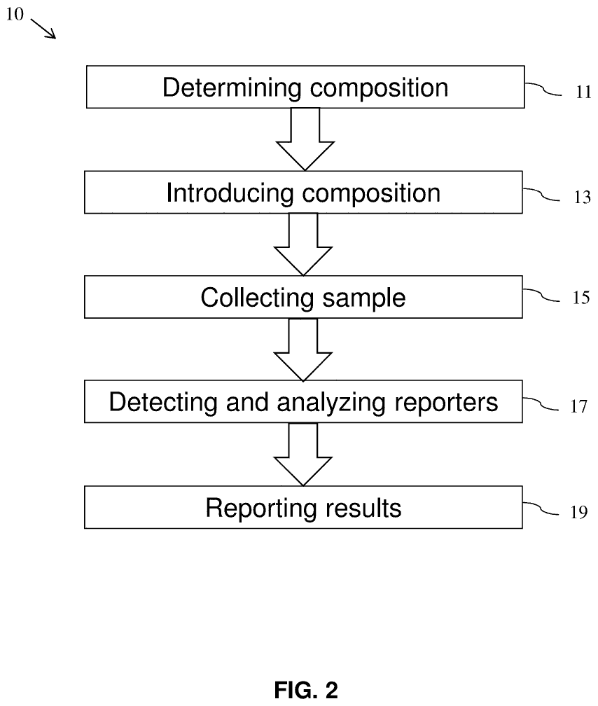

FIG. 2 illustrates a method 10 of the invention. A composition is designed 11 based on protease activity. The composition is administered 13 to a subject. Any suitable introduction method may be used. For example, the composition may be introduced to a subject by intravenous injection, ingestion, and inhalation, among other methods. Activity sensors in the composition collect in a desired tissue. In the tissue, the cleavage sites are cleaved by active proteases and liberate reporters from the activity sensors. After a specified time period, such as about one hour, a sample is collected 15 from the subject. The sample collected may be any suitable sample. For example, the sample collected is a urine sample. The sample is analyzed to detect the liberated reporters cleaved by the active proteases in the sample 17.

A set of proteases active together in tissue at any given time provides a sensitive marker along a continuum of health and may indicate a disease and a stage of the disease. Also, because the activity sensors provide an excess number of reporters for the enzymatic cleavage, the presence of reporters or detectable analytes in a sample from the body may be measured quantitatively to give a measure of rate of activity of the proteases. The rates of activity of the enzymes collectively may serve as an instantaneous measure of rate of progression of the disease.

The rate of activity of the disease may be detected by compositions of the invention. For example, fibrosis is a disease that has a scale of progression. At one end of the scale, a patient may be considered untreatable and have “burnt out NASH” because tissue is fibrotic and does not exhibit enzymatic activity. A subject with fibrosis may be administered a composition of the invention in order to detect enzymatic activity in the fibrotic tissue and thus determine whether the subject will be responsive to treatment. If tissue biopsy or liver stiffness were instead used as detection methods, the tests would indicate that the subject has fibrotic tissue, but no information about the enzymatic activity would be provided. Thus, the invention provides personalized medicine methods that include multiple intra-patient measurements over time for the detection of activity that would not be shown otherwise. As such, the invention allows for detection of orthogonal disease metrics instead of merely the presence or absence of the disease.

Any suitable method may be used for detection and analysis of the liberated reporters. For example, methods of detection include mass spectrometry, chromatography, volatile organic compounds (VOC), enzyme immunoassay, such as enzyme-linked immunosorbent assay (ELISA), imaging, such as magnetic imaging, breath analyzer, and single molecule, paper diagnostic, nucleic acid coding, imaging, and control probes. For example, the reporters may be barcoded with a fluorescent metal nanoparticle, and the fluorescent metal nanoparticle is detected by imaging over the area of the body or tissue where the reporters collect. For example, the sample is analyzed by mass spectrometry (which assays for a mass to charge ratio of polypeptides cleaved from the activity sensors, with results shown by peaks in the mass spectra).

In a preferred embodiment, the method of detection is ELISA. Using ELISA, antigens from the sample are attached to a surface, such as wells of a plate. A buffer containing unrelated protein may be used to block free sites e.g., in the wells of the plate. Then, a specific antibody is applied so it can bind to the antigen. This antibody is linked to an enzyme. In the final step, a substance containing the substrate of the enzyme is added, which generates a detectable signal, commonly a color change in the substrate.

The result of detection and analysis provides a therapeutic response profile of the subject, which may then be reported to the subject 19. For example, results may indicate presence of a disease or condition, staging of a disease, and/or rate of progression of a disease in the tissue (e.g., peaks in mass spectra). The report may further describe the therapeutic profile of a subject and may indicate a physiological condition of the subject. The report may be used for diagnosis, monitoring, and/or treatment of the subject and for qualification and stratification of the patient in a clinical trial.

The present invention provides methods of determining a therapeutic response profile. A composition is introduced into a body of a subject. The composition comprises an activity sensor comprising a plurality of reporters liberated through enzymatic cleavage, which occurs in a tissue of the body when enzymes associated with known activity of a therapeutic intervention are present in the body. One or more reporters are detected to determine the therapeutic response profile of the subject. The therapeutic response profile may further be used to support a clinical trial. For example, the therapeutic response profile may indicate the individual suffers from a particular condition or disease. Furthermore, the therapeutic response profile may be used to stratify prospective or active clinical trial participants. For example, the therapeutic response profile may indicate that an individual is more likely to respond to a particular course of treatment or drug.

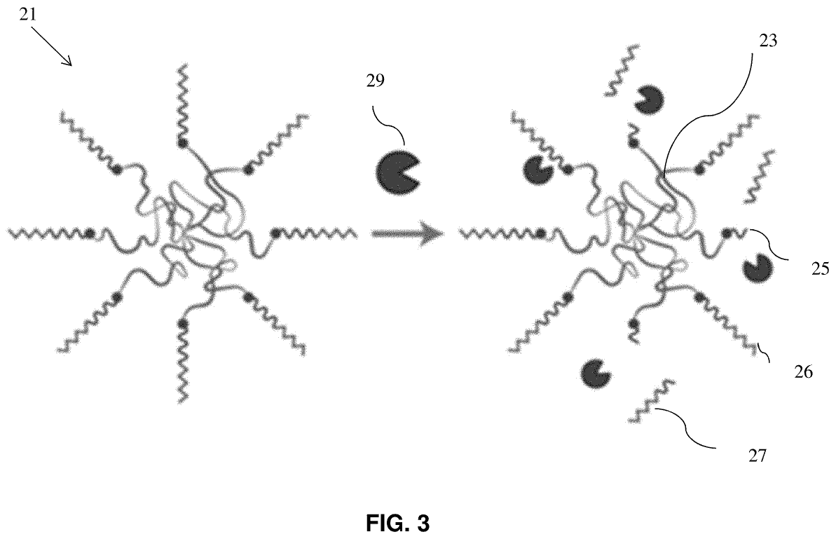

FIG. 3 shows an activity sensor 21 according to certain embodiments. Compositions of the present invention comprises a plurality of activity sensors. Each activity sensor 21 comprises a carrier or core 23 with a plurality of reporters 26 attached thereto. The reporters 26 are susceptible to cleavage and liberation by enzymes. The liberated reporters 27 are indicative of enzymatic activity associated with particular disease or condition. As such, detection of the liberated reporters at multiple time points provide for longitudinal monitoring. Detected quantities of the liberated reporters may be correlated to a disease state or drug response in the subject. For example, where a disease involves the expression of proteases (e.g., granzyme B), detecting lower quantities of peptide fragments (liberated reporters 27) over time may indicate disease regression and positive drug response.

In clinical trial support methods, the liberated reporters may indicate healthy or normal enzymatic behavior and thus qualify a participant for Phase I. The liberated reporters may indicate enzymatic activity associated with a disease and thus qualify a patient for subsequent phases of the clinical trial, i.e., Phases II-IV. For longitudinal monitoring, the activity sensors 21 may be administered multiple times with associated sample collection and assay. The activity sensors 21 include peptide chains linked to a carrier, such that the detected signal is provided by fragments of the peptide chains that are released by proteases for which expression is upregulated at the location in the body when affected by the condition. Each carrier may include multiple copies of only one peptide chain such that each activity sensor is specific to one protease. The activity sensors as a set may report the activity of multiple proteases. Using such a set of activity sensors when the condition is a disease and, when the disease presents in a person: at least some of the multiple proteases are upregulated and at least one of the multiple proteases is not specifically upregulated.

Furthermore, the liberated reporters may indicate enzymatic activity associated with a particular stage of a disease. Detection of reporters indicative of a disease stage enable qualification and stratification of the participant in the clinical trial. The participant may be given a specified dose of a new drug and then the treatment may be monitored to show effectiveness.

The carrier may be any suitable carrier or core. In a preferred embodiment, the carrier or core is a polyethylene glycol (PEG) polymer. For example, the carrier may comprise a 30 kDa to 40 kDa PEG scaffold of covalently linked PEG subunits. For example, the carrier may be selected from PEG-MAL and 40 kDa eight-arm PEG. Use of the PEG carrier provides better bioavailability, circulation time, and safety. Linking peptides to PEG allows for the peptides to withstand clearance by the kidneys within minutes of administration. As such, the activity sensors and reporters are detectable in a urine sample, which may be collected from the clinical trial participant shortly after administering the composition. Furthermore, such linking with PEG creates a larger molecular structure and limits uptake to the cells. PEG is not immunogenic or toxic, thereby allowing for less frequent administration and lower doses.

When the carrier 21 is subjected to a protease 29, the protease 29 cleaves the reporter 26 at a cleavage site 25. The liberated reporter is then a detectable analyte 27. The liberated reporters or analytes may be any suitable material and preferably are amino acids, peptides, or polypeptides. In preferred embodiments, the reporters 26 of the activity sensors 21 are polypeptides that include cleavage sites 25 of proteases and are cleaved by the proteases to release the reporters or detectable analytes 27. By including cleavage sites 25 in the polypeptide reporters 26, the activity sensors 21 may be designed to report the activity of any proteases. Any suitable protease or protease category may be queried by the activity sensors 21, including, for example, cysteine proteases, aspartic proteases, serine proteases, threonine proteases, or metalloproteases. Further, each reporter and cleavable linker may comprise a polypeptide susceptible to cleavage by a protease known to be associated with a specific disease or condition associated with treatment options tested in a clinical trial.

Although enzymatic cleavage is discussed herein, other methods may be used to cleave the reporters. Cleavage by light and chemical cleavage are non-limiting examples of cleavage that do not occur from enzymatic activity. The reporter may be any suitable reporter. In certain embodiments, the reporter may be barcoded for detection purposes. In preferred embodiments, the reporter is engineered to be a substrate cleavable by a protease associated with a particular disease or condition associated with treatment options tested in a clinical trial.

The present invention provides methods of longitudinal monitoring of disease changes. A composition is introduced into a subject and measurements are made over time. The composition comprises an activity sensor comprising a plurality of reporters liberated through enzymatic cleavage. Enzymatic cleavage occurs in a tissue of the body when enzymes associated with known activity of a therapeutic intervention are present in the body. One or more liberated reporters are detected to determine a therapeutic response profile of the prospective or active clinical trial participant.

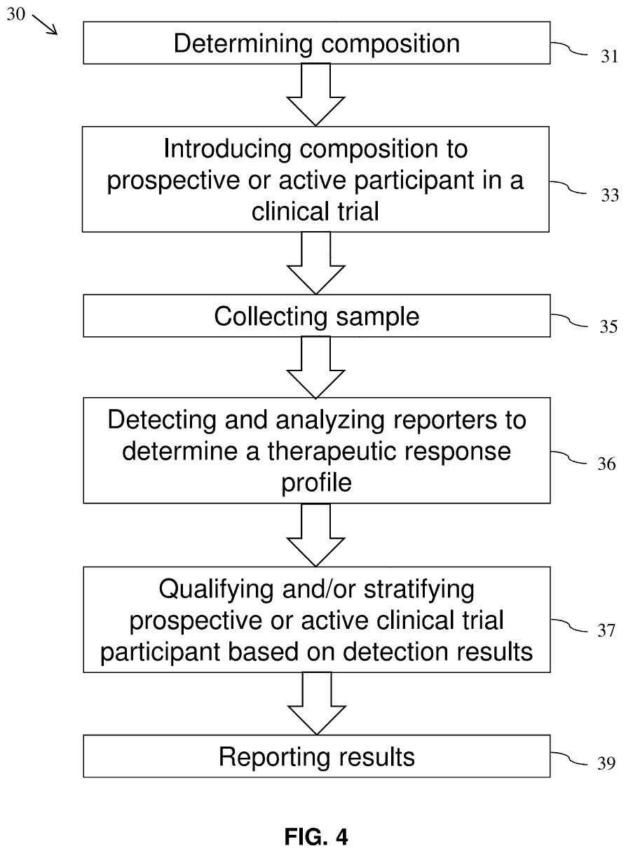

FIG. 4 diagrams a method 30 according to certain embodiments for clinical trial support. In the method 30, a composition of the disclosure is used to qualify patients for a clinical trial or determine drug efficacy during the conduct of a clinical trial. The method 30 includes determining 31 a composition the readout of which reports a disease-relevant physiological state of subject or participant (active or prospective) in a clinical trial. Compositions as described herein may be administered or introduced 33 to a prospective or active clinical trial participant. For example, composition may be introduced to the prospective or active clinical trial participant by intravenous injection. Where the composition includes the activity sensors, the activity sensors will collect in a desired tissue. In the tissue, the cleavage sites are cleaved by active proteases. A set of proteases active together in tissue at any given time provides a sensitive marker of along a continuum of health and may indicate a physiological state or condition, such as a disease or a pre-disease state.

Any suitable delivery route may be used in the clinical trial support method 30. For example, routes of delivery include intravenous, aerosol inhalation, subcutaneous sustained release, oral ingestion, and transdermal delivery. As a non-limiting example, aerosolized probes may retain the ability to detect protease activity and aerosol delivery of the probes may significantly accumulate in lung tumors. In a preferred embodiment, the route of delivery is intravenous delivery by an injection.

In the clinical trial support method 30, a sample is then collected from the prospective of active participant in the clinical trial 35. For example, the sample is a urine sample. The sample may be any suitable sample from a subject. For example, the sample may be a urine sample, a breath sample, a sweat sample, or a tissue sample. Furthermore, the sample may be collected by any suitable method. For example, the sample may be excreted from the body, the sample may be obtained by liquid or tissue biopsy, and the sample may be imaged using imaging methods.

In the clinical trial support method 30, the sample is then tested using a detection method to detect and analyze reporters in the sample to determine a therapeutic response profile 36. Because the activity sensors provide an excess number of substrates for the enzymatic cleavage, the presence of reporters or detectable analytes in a sample from the body may be measured to give a measure of the protease activity or therapeutic response profile.

In the clinical trial support method 30, detection of the liberated reporters may be by any suitable detection method. The liberated reporters of the present invention may be detected by any suitable method of detection. For example, methods of detection of the activity barcodes include mass spectrometry, chromatography, volatile organic compounds (VOC), enzyme immunoassay, such as enzyme-linked immunosorbent assay (ELISA), imaging, such as magnetic imaging, breath analyzer, and single molecule, paper diagnostic, nucleic acid coding, imaging, and control probes. In a preferred embodiment, the method of detection is enzyme immunoassay.

The therapeutic response profile may then be used to qualify or stratify the prospective or active clinical trial participant 37. The therapeutic response profile may indicate that the prospective or active clinical trial participant suffers from a particular physiological state or condition or indicate that the prospective or active clinical trial participant may respond to a particular course of treatment. In certain embodiments, the results of the therapeutic response profile and potential clinical trial participant qualification and/or stratification may be reported 39. The therapeutic response profile may indicate a physiological state in the prospective or active clinical trial participant. In an embodiment, the physiological state comprises a disease and the therapeutic response profile further indicates staging of the disease. In certain embodiments, the physiological state comprises a pre-disease state. The pre-disease state may qualify the prospective or active clinical trial participant for a clinical trial.

In an embodiment of the clinical trial support method 30, a prospective or active clinical trial participant is identified who will respond to a drug or therapeutic treatment based on the therapeutic response profile. In certain embodiments, the therapeutic response profile is used to monitor a clinical trial participant's response to a drug or course of treatment by determining the therapeutic response profile before and after administering the drug or course of treatment. In an aspect, the therapeutic response profile indicates responsiveness of the clinical trial participant to the administered drug or course of treatment. In other aspects, the therapeutic response profile indicates responsiveness of the clinical trial participant to a specific dose of the drug or course of treatment. In other embodiments, the therapeutic response profile indicates efficacy or effectiveness of the drug or course of treatment for the clinical trial participant.

Furthermore, participants in the clinical trial may be stratified. Stratification is the partitioning of participants based on a factor other than the treatment given. For example, stratification may allocate participants into subgroups with experimental conditions, fitness, gender, age, or other demographic factors. Stratification may also be used to identify participants that are more likely to respond to a particular drug or treatment.

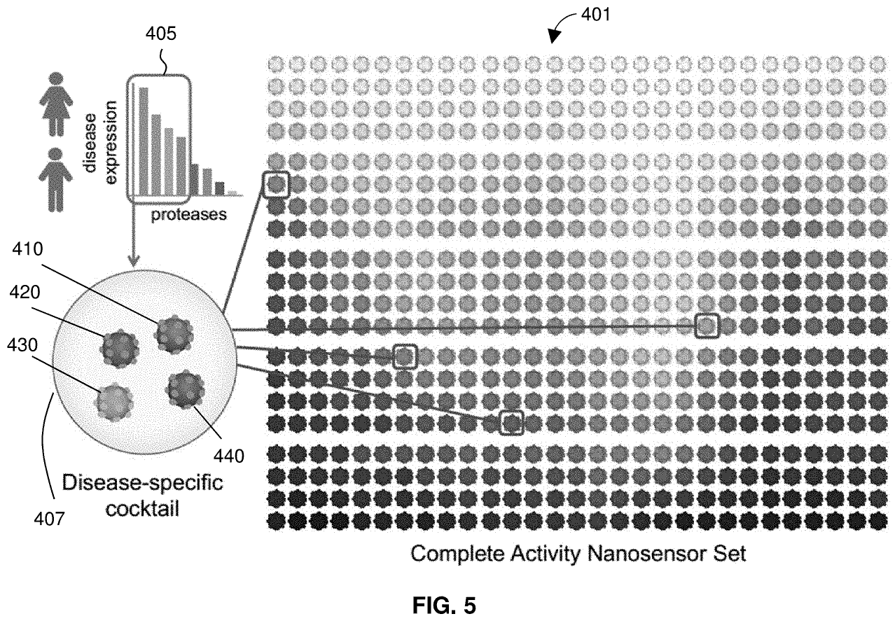

FIG. 5 shows design of the composition according to the present invention. The composition 407 preferably includes a plurality of activity sensors. Each activity sensor has a plurality of reporters. The reporters for each activity sensor are cleavable by proteases found to be expressed specifically in the disease of interest. Proteases are enzymes that perform significant biological processes. A complete set 401, or library, of the activity sensors may be designed or made, each associated with detection of a specific protease. As such, each activity sensor, or nanosensor, in the library is associated with a particular enzyme. Therefore, each activity sensor in the library is characteristic of a particular disease or condition. In the present invention, the proteases associated with the reporters are characteristic of a particular disease or condition to be treated during a clinical trial. As such, a composition used in a clinical trial may be designed to include detection of a plurality of enzymes.

For a given disease, patient samples may be used to create a profile 405 of a level of expression of each protease associated with the disease. Based on those proteases that are expressed specifically in the disease, particular activity sensors, such as 410, 420, 430, and 440, are selected from the set 401 for inclusion in a composition 407. For example, activity sensors 410, 420, 430, and 440 may be directed to proteases associated with a tumor in lung cancer. As such, activity sensors 410, 420, 430, and 440 are included in a composition 407 directed to detection of tumors in lung cancer, which may be used in qualifying participants in a clinical trial directed to treatment of lung cancer.

The activity sensors of the present invention detect dysregulated proteases associated with disease. Activity sensors may be formulated from any suitable material and preferably are PEG and mass-barcoded protease substrates. The activity sensors each comprise a plurality of detectable reporters. In preferred embodiments, the reporters are peptide substrates designed to be cleaved by specific proteases. In an embodiment, the signals the sensors produce may be barcoded onto the activity sensors. Because the activity sensors are engineered, the readout may be by any suitable method. For example, the readout may be by mass spectroscopy, lateral flow, or ELISA. A bar-coded activity sensor cocktail, or composition, is assembled for each disease of interest by looking at proteases for the disease expression. The activity sensor cocktail is engineered and administered to a subject. A sample is collected from the subject, such as a urine sample, after a suitable time period, e.g. about 1 hour. The sample is then analyzed to detect the barcodes and provide results to the subject.

In certain embodiments, the composition comprises a plurality of activity sensors, each activity sensor programmed with reporters susceptible to cleavage by specific enzymes. The enzymes may be any suitable enzyme. In particular embodiments, the specific enzyme is associated with normal enzymatic activity in a tissue or healthy tissue, thereby indicating a baseline enzyme activity level of the subject. Such an indication may be used to qualify participants for Phase I of a clinical trial. The specific enzyme may be associated with a particular disease or condition, which may be used to qualify patients for subsequent phases of a clinical trial, i.e., Phases II-IV.

Furthermore, the specific enzyme may be indicative of one or more comorbidities to a disease or condition. Other detectable reporters are associated with proteases are indicative of a disease or condition different from the target disease or condition, which may be a comorbidity or related disease producing effects similar to effects of the target disease. By including the “off-target” protease detection in the composition, a more specific therapeutic response profile may be determined, as detection is more sensitive and less likely to indicate a false positive result for the specified disease. This enables further stratification of the participant in the clinical trial. A subset of the clinical trial may be monitoring participants with comorbidities to determine effects of treatment in participants with comorbidities.

In an aspect of the invention, the composition comprises a plurality of activity sensors, each activity sensor programmed with reporters susceptible to a specific enzyme. The composition may comprise thousands of activity sensors, wherein each activity sensor is a nanosensor. Of the activity sensors in the composition, hundreds may be activity sensors directed to activity detection of a first particular enzyme, hundreds may be activity sensors directed to activity detection of a second particular enzyme, hundreds may be activity sensors directed to activity detection of a third particular enzyme, etc. As such, the composition is engineered for detection of a specific condition and may be designed to optimally stratify a participant in a clinical trial.

As an example, in the clinical trial support method 30, a clinical trial may relate to a new drug or treatment method of nonalcoholic steatohepatitis (NASH). The specific enzyme is indicative of NASH, and a composition directed to detection of NASH includes activity sensors with reporters susceptible to cleavage by enzymes associated with NASH. In the composition, there may be a plurality of activity sensors susceptible to protease FAP associated with NASH, a plurality of activity sensors susceptible to protease MMP2 associated with NASH, and a plurality of activity sensors susceptible to protease ADAMTS2 associated with NASH. Further, another specific enzyme is indicative of comorbidities to NASH, such as obesity and Type 2 Diabetes. As such, the composition directed to detection of NASH should also include activity sensors with a plurality of reporters susceptible to cleavage by enzymes associated with obesity and a plurality of reporters susceptible to cleavage by enzymes associated with Type 2 Diabetes.

By providing specific enzymes to indicate a baseline enzyme activity level for the subject, probe for a specific disease or condition, and probe for comorbidities of that specific disease or condition, the present invention provides a therapeutic profile for the subject.

The present invention thus provides methods for determining therapeutic response profiles for potential or active clinical trial participants. Methods and compositions of the invention are useful in clinical trial design, predicting patients and cohorts of patients most likely to respond to therapy, planning for qualification and stratification of participants, and for longitudinal patient monitoring on drugs following regulatory approval. The compositions according to the present invention are designed to detect and screen for particular enzymatic activity indicative of a disease or condition to be treated during the clinical trial. This provides a quick, simple method of qualifying or stratifying participants without having to undergo expensive, time-consuming testing, such as liquid biopsy, tissue biopsy, or other methods. Indication of the disease presence or absence, rate of activity of the disease, and staging of the disease are used to qualify and stratify participants in the clinical trial. For example, the detected reporters may indicate the participant has a particular disease, which is used to qualify the participant. The detected reporters may further be used to stratify the participant. For example, the disease may be in an initial stage and the participant would then be stratified with other participants in initial stages of the disease.

In the clinical trial support method 30, activity sensors are used to measure disease-associated activity within the body such that levels of such activity measured using the sensors shows disease progression or that treatment is being effective. The activity sensors may be given over time to monitor a subject's response to treatment and to show whether or not the treatment is effectively treating the disease in the subject. In the clinical trial support method 30, different drugs or combinations of drugs can be studied in different subjects or at different times to identify drugs or combinations that work well for treating a condition. The activity sensors provide a marker of health/ disease progression and provide the marker very quickly (detecting a signal in a sample from the subject may be within hours of administering the activity sensors). That activity sensors can also specifically report the activity of multiple enzymes including ones for which expression is dysregulated in the disease state and others that are specific to co-morbidities. The activity sensors thus provide an informative signature of response to treatment. Specific aspects of the activity or progression of the disease may be detected, such as growth rate of a tumor.

Using compositions and methods described herein, a large population may be screened quickly and effectively to detect or rule out a particular disease state. Similarly, a cohort of disease-affected subjects can be assayed to detect and quantify an effect of a drug on the disease. Compositions and methods of the disclosure thus provide tools for supporting clinical trials. In particular, the invention allows for the rapid and inexpensive qualification of subject populations. The invention also provides methods and compositions useful for the rapid determination of subjects' response to a drug.

Compositions of the disclosure are particularly useful for patient qualification. The compositions can be administered in a minimally invasive manner and can provide informative results rapidly (e.g., in a matter of hours). Thus, the compositions provide a tool for clinical trial planners for screening large populations of subjects to identify a prospective cohort of trial participants.

In certain aspects, the invention provides methods for determining treatment efficacy. Activity sensors are administered to a subject who is receiving, or will receive, a treatment for a condition. The activity sensors release a signal at a location in the body affected by the condition. A sample is obtained from the subject, and an assay is performed to detect the signal in the sample. Methods include reporting an effect of the treatment on the condition based on the detected signal. A plurality of subjects may be monitored over time for response to a plurality of different treatments. The condition may be a disease (e.g., a disease of the liver such as NASH) and the plurality of different treatments may each comprise a drug or combination of drugs. The method may include identifying an treatment with efficacy in treating the disease.

In certain embodiments, the signal is provided by fragments of the activity sensors that are released by enzymes (e.g., extracellular proteases) for which expression is upregulated at the location in the body when affected by the condition. Using the methods for longitudinal monitoring, it may be found that a decrease in certain fragments from the subject (compared to a control receiving a placebo) is indicative of efficacy of the treatment as to the condition.

For longitudinal monitoring, the activity sensors may be administered at a second time point followed by an additional sample collection and assay, such that reporting the effect of the treatment includes progress or remission over time for the subject.

Steps of the methods may be performed for each of a plurality of unique subjects, each of the unique subject receiving one of a plurality of trial treatments each comprising one or a combination of drugs. The methods can include providing a report or populating a database with efficacy of the trial treatments. The methods may include providing a longitudinal analysis of efficacy of a plurality of combinations of trial drugs for treatment of a disease.

In some embodiments, the activity sensors include peptide chains linked to a carrier, such that the detected signal is provided by fragments of the peptide chains that are released by proteases for which expression is upregulated at the location in the body when affected by the condition. In certain embodiments, each carrier includes multiple copies of only one peptide chain such that each activity sensor is specific to one protease. The activity sensors as a set may report the activity of multiple proteases. Using such a set of activity sensors when the condition is a disease and, when the disease presents in a person: at least some of the multiple proteases are upregulated and at least one of the multiple proteases is not specifically upregulated. When the condition comprises an inflammatory condition, the administration step may include causing the activity sensors to localize to a site of inflamed tissue. Preferably, the activity sensors comprise peptide chains linked to a carrier, such that the signal comprises fragments of the peptide chains that are released by proteases for which expression is upregulated at the site of the condition, and in which the assay comprises mass spectrometry or an affinity assay to detect or quantify the fragments in the sample.

Compositions and methods of the invention are useful for determining treatment efficacy within, for example, the context of a drug study or longitudinal study. Activity sensors are administered to a subject who is receiving, or will receive, a treatment. The activity sensors release a signal at a location in the body affected by a disease or condition. A sample is obtained from the subject, and an assay is performed to detect the signal in the sample. Methods include reporting an effect of the treatment on the condition based on the detected signal. A plurality of subjects may be monitored over time for response to a plurality of different treatments. The disease may be a disease of the liver such as NASH and the different treatments may each comprise a drug or combination of drugs. Thus methods and compositions of the disclosure may be used to identify new combinations or dosages of drugs that are effective in treating a disease.

One of skill in the art would know what peptide segments to include as protease cleave sites in an activity sensor of the disclosure. One can use an online tool or publication to identify cleave sites. For example, cleave sites are predicted in the online database PROSPER, described in Song, 2012, PROSPER: An integrated feature-based tool for predicting protease substrate cleavage sites, PLoSOne 7(11):e50300, incorporated by reference. Reproduced below is a set of exemplary protease substrates for a variety of significant protease. In the sequences shown below, the vertical bar shows the cleavage site, and forms no part of the sequence. Any of the compositions, structures, methods or activity sensors discussed herein may include, for example, any of the sequences below as cleavage sites, as well as any further arbitrary polypeptide segment to obtain any desired molecular weight. To prevent off-target cleavage, one or any number of amino acids outside of the cleavage site may be in a mixture of the D and/or the L form in any quantity.

Aspartic protease HIV-1 retropepsin (A02.001)

| | |
|---|---|
| A02.001: | SSTSISWYS (SEQ ID NO: 1) |
| A02.001: | PCIQIAESE (SEQ ID NO: 2) |
| A02.001: | DDEEIIELA (SEQ ID NO: 3) |
| A02.001: | VLEQIVVTS (SEQ ID NO: 4) |
| A02.001: | QVVQIVVLD (SEQ ID NO: 5) |

Cysteine protease Cathepsin K (C01.036)

| | |
|---|---|
| C01.036: | KSIQIEIQE (SEQ ID NO: 6) |
| C01.036: | KDFAIAEVV (SEQ ID NO: 7) |
| C01.036: | TSYAIGYIE (SEQ ID NO: 8) |
| C01.036: | LKVAIGQDG (SEQ ID NO: 9) |
| C01.036: | FCLHIGGLS (SEQ ID NO: 10) |

Calpain-1 (CO2.001)

| | |
|---|---|
| CO2.001: | WMDFIGRRS (SEQ ID NO: 11) |
| CO2.001: | SATAIAVNP (SEQ ID NO: 12) |
| CO2.001: | RELGILGRH (SEQ ID NO: 13) |

Caspase-1 (C14.001)

| | |
|---|---|
| C14.004: | DEGDISLDG (SEQ ID NO: 14) |
| C14.004: | DETDIMAKL (SEQ ID NO: 15) |
| C14.004: | EECDIAAEG (SEQ ID NO: 16) |

Caspase-3 (C14.003)

| | |
|---|---|
| C14.003: | AEVDIGDDD (SEQ ID NO: 17) |
| C14.003: | DRHDIGTSN (SEQ ID NO: 18) |
| C14.003: | VEVDIAPKS (SEQ ID NO: 19) |

Caspase-7 (C14.004)

| | |
|---|---|
| C14.004: | DQTDIGLGL (SEQ ID NO: 20) |
| C14.004: | DSIDISFET (SEQ ID NO: 21) |
| C14.004: | DDVDITKKQ (SEQ ID NO: 22) |

Caspase-6 (C14.005)

| | |
|---|---|
| C14.005: | VEMDIAAPG (SEQ ID NO: 23) |
| C14.005: | VSWDISGGS (SEQ ID NO: 24) |
| C14.005: | EETDIGIAY (SEQ ID NO: 25) |

Caspase-8 (C14.009)

| | |
|---|---|
| C14.003: | VETDIKATV (SEQ ID NO: 26) |
| C14.003: | GSSDIPLIQ (SEQ ID NO: 27) |
| C14.003: | DDADIYKPK (SEQ ID NO: 28) |

Metalloprotease Matrix metallopeptidase-2 (M10.003)

| | |
|---|---|
| M10.003: | HISS ILIKL (SEQ ID NO: 29) |
| M10.003: | DPNNILLND (SEQ ID NO: 30) |
| M10.003: | DLSDILTAA (SEQ ID NO: 31) |
| M10.003: | FSAYIIKNS (SEQ ID NO: 32) |
| M10.003: | EALPILLVR (SEQ ID NO: 33) |

Matrix metallopeptidase-9 (M10.004)

| | |
|---|---|
| M10.004: | QQGAIIGSP (SEQ ID NO: 34) |
| M10.004: | GPPGIIVIG (SEQ ID NO: 35) |
| M10.004: | MDIAIIHHP (SEQ ID NO: 36) |
| M10.004: | FFKNIIVTP (SEQ ID NO: 37) |
| M10.004: | GPLGIARGI (SEQ ID NO: 38) |

Matrix metallopeptidase-3 (M10.005)

| | |
|---|---|
| M10.005: | HLGGIAKQV (SEQ ID NO: 39) |
| M10.005: | VWAAIEAIS (SEQ ID NO: 40) |
| M10.005: | GPLGIARGI (SEQ ID NO: 41) |
| M10.005: | ESGDIYKAT (SEQ ID NO: 42) |

Matrix metallopeptidase-7 (M10.008)

| | |
|---|---|
| M10.008: | VAQDILNAP (SEQ ID NO: 43) |
| M10.008: | SPDAILQNP (SEQ ID NO: 44) |
| M10.008: | PPLKILMHS (SEQ ID NO: 45) |
| M10.008: | GPHLILVEA (SEQ ID NO: 46) |

Serine protease Chymotrypsin A
(cattle-type) (S01.001)

| | |
|---|---|
| S01.001: | VGPNILHGV (SEQ ID NO: 47) |
| S01.001: | GGGNIKIGP (SEQ ID NO: 48) |

Granzyme B (Homo sapiens-type) (S01.010)

| | |
|---|---|
| S26.010: | LSTAIRFVV (SEQ ID NO: 49) |
| S26.010: | VTEDIVDIN (SEQ ID NO: 50) |
| S26.010: | SALAITTVY (SEQ ID NO: 51) |

Elastase-2 (S01.131)

| | |
|---|---|
| S01.131: | QELIISNAS (SEQ ID NO: 52) |
| S01.131: | QELIISNAS (SEQ ID NO: 53) |
| S01.131: | WELIISNAS (SEQ ID NO: 54) |

Cathepsin G (S01.133)

| | |
|---|---|
| S01.133: | SGNYIATVI (SEQ ID NO: 55) |
| S01.133: | SIQMINVAE (SEQ ID NO: 56) |
| S01.133: | QQNYIQNSE (SEQ ID NO: 57) |

Thrombin (S01.217)

| | |
|---|---|
| S01.217: | SILRILAKA (SEQ ID NO: 58) |
| S01.217: | KFQRIAITG (SEQ ID NO: 59) |
| S01.217: | AEPKIMHKT (SEQ ID NO: 60) |
| S01.217: | TIPRIAAIN (SEQ ID NO: 61) |

Plasmin (S01.233)

| | |
|---|---|
| S01.233: | AEFRIHDSG (SEQ ID NO: 62) |
| S01.233: | RRKRIIVGG (SEQ ID NO: 63) |
| S01.233: | AMSRIMSLS (SEQ ID NO: 64) |

Glutamyl peptidase I (S01.269)

| | |
|---|---|
| S01.269: | PEPEIQLKM (SEQ ID NO: 65) |
| S01.269: | QSKEIAIHS (SEQ ID NO: 66) |
| S01.269: | KLKEIASRS (SEQ ID NO: 67) |

Furin (S08.071)

| | |
|---|---|
| S08.071: | RAKRISPKH (SEQ ID NO: 68) |
| S08.071: | RKKRISTSA (SEQ ID NO: 69) |

Signal peptidase I (S26.001)

| | |
|---|---|
| S26.001: | SAMAIADSN (SEQ ID NO: 70) |
| S26.001: | TLLAININE (SEQ ID NO: 71) |

Thylakoidal processing peptidase (S26.008)

| | |
|---|---|
| S01.269: | QAEEITYEN (SEQ ID NO: 72) |
| S01.269: | DVIDIMSKE (SEQ ID NO: 73) |

Signalase (animal) 21 KDa component (S26.010)

| | |
|---|---|
| S26.010: | EVLAITPPA (SEQ ID NO: 74) |
| S26.010: | APVPIGTAW (SEQ ID NO: 75) |

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A longitudinal monitoring method for drug response, the method comprising:
monitoring change in disease condition in a subject who has received a therapeutic treatment for the disease by making measurements at multiple points over time wherein each measurement comprises:
administering, to the subject, activity sensors comprising detectable reporters that are released from the activity sensors in proportion to a state of the disease;
detecting the reporters in a sample from the subject;
correlating quantities of the reporters to disease state.

2. The method of claim 1, wherein the measurements at multiple time points reveal progression or regression of the disease in the patient in response to the therapeutic treatment or after the conclusion of therapeutic treatment.

3. The method of claim 1, further comprising predicting the benefit of the therapeutic treatment for the patient with the disease based on the measurements at multiple time points.

4. The method of claim 1, further comprising recommending a change in the therapeutic treatment based on the measurements at multiple time points.

5. The method of claim 1, wherein the measurements at multiple time points reveal a rate of progression or regression of the disease in the patient.

6. The method of claim 1, further comprising determining a stage of the disease in the subject.

7. The method of claim 1, wherein the disease comprises an allergy, cardiovascular disease, degenerative disease, neurological disease, dietary disease, hereditary disease, immune disease, inflammatory disease, infectious disease, metabolic disease, cancer, organ disease, pregnancy, muscle injury, or trauma disease.

8. The method of claim 1, wherein the disease is cancer.

9. The method of claim 8, further comprising correlating measured levels of the detectable reporter in the sample to a stage of the cancer and providing a report describing a stage of the cancer in the subject.

10. The method of claim 8, further comprising monitoring how a tumor in the subject responds to a drug or therapeutic treatment.

11. The method of claim 1, wherein the plurality of detectable reporters are substrates for a plurality of enzymes, wherein at least some of the enzymes exhibit differential activity levels in tissue affected by the disease, compared to healthy tissue or cells.

12. The method of claim 11, wherein one of the enzymes does not exhibit differential activity levels in tissue affected by the disease and is used to indicate a baseline enzyme activity level.

13. The method of claim 11, wherein one of the enzymes is indicative of one or more comorbidities to the disease.

14. The method of claim 1, wherein each activity sensor comprises:
a carrier comprising one or a plurality of molecular subunits and a plurality of the detectable reporters, each linked to the carrier by a cleavable linker containing a cleavage site of an enzyme.

15. The method of claim 14, wherein the carrier comprises a 20 to 60 kDa scaffold of poly ethylene glycol (PEG) subunits, a tissue- or organ-specific antibody, or alternative synthetic carrier.

16. The method of claim 14, wherein each reporter and cleavable linker comprises a polypeptide susceptible to cleavage by a protease known to be associated with a specific disease or condition.

17. The method of claim 1, further comprising determining efficacy of the therapeutic treatment for the disease by detecting a decrease the reporters in the subject as compared to a second subject administered a placebo.
